SEQUENCE LISTING (9 sequences)

SEQ ID NO: 1 - DNA, 23 bp, Artificial Sequence
  Oligonucleotide
tctatgacctcgccctccacaaa

SEQ ID NO: 2 - DNA, 27 bp, artificial sequence
  Synthetic oligonucleotide
gaacggtgtcttcaggttggtatttca

SEQ ID NO: 3 - DNA, 24 bp, Artificial Sequence
  Synthetic oligonucleotide
aacctggagtttgtgccagggttt

SEQ ID NO: 4 - DNA, 24 bp, Artificial Sequence
  Synthetic oligonucleotide
tgaacttcaccttccctccaacca

SEQ ID NO: 5 - DNA, 26 bp, Artificial Sequence
  Synthetic Oglionucleotide
cagtggcagtctcaggttaagaagga

SEQ ID NO: 6 - DNA, 21 bp, Artificial Sequence
  Synthetic oglionucleotide
cgctactgcaggtgtgagcaa

SEQ ID NO: 7 - DNA, 24 bp, Artificial Sequence
  Synthetic oglionucleotide
aatactctgtctggatcggtggct

SEQ ID NO: 8 - DNA, 24 bp, Artificial Sequence
  Synthetic oglionucleotide
acgagtcagagctttggctaggaa

SEQ ID NO: 9 - DNA, 1438 bp, Homo sapiens
gctcatccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacg

(19) United States
(12) Patent Application Publication
(10) Pub. No.: US 2012/0301443 A1
(43) Pub. Date: Nov. 29, 2012

(54) METHODS FOR DEVELOPING ENDOTHELIAL CELLS FROM PLURIPOTENT CELLS AND ENDOTHELIAL CELLS DERIVED

(75) Inventors: Shahin Raffi, New York, NY (US); Daylon James, New York, NY (US)

(73) Assignee: CORNELL UNIVERSITY, Ithaca, NY (US)

(21) Appl. No.: 13/519,473

(22) PCT Filed: Dec. 23, 2010
(86) PCT No.: PCT/US10/61970

§ 371 Date: Jun. 27, 2012

Related U.S. Application Data
(60) Provisional application No. 61/290,667, filed on Dec. 29, 2009.

(51) Int. Cl. C12N 5/071; A61P 35/00; C12N 5/10; A61K 35/12

(52) U.S. Cl. 424/93.7; 435/366

(57) ABSTRACT

Disclosed herein is a method for developing human endothelial cells (ECs) from human embryonic stem cells (ESCs). The method is based on inhibition of TGF signaling following mesoderm induction and during vascular differentiation of hESC-derived cells. Also disclosed herein is a substantially pure and stable population of ECs that maintains a high degree of proliferation and phenotypic homogeneity for extended culture periods. Related pharmaceutical compositions and therapeutic methods are also disclosed. A reporter hESC line useful for tracking the development of ECs is also provided.

### CROSS REFERENCE TO RELATED APPLICATION

This application claims the benefit of U.S. Provisional Application No. 61/290,667, filed on Dec. 29, 2009, the entire contents of which are incorporated herein by reference.

### FIELD OF THE INVENTION

This disclosure generally relates to generation of human endothelial cells (ECs) from human embryonic stem cells (ESCs) in culture. More specifically, this disclosure relates to a method of developing human ECs from human ESCs based on inhibition of TGFβ signaling following mesoderm induction and during vascular differentiation of hESC-derived cells. ECs developed by such method, and related pharmaceutical compositions and therapeutic methods are also disclosed.

### BACKGROUND ART

Human embryonic stem cells (hESC), which self-renew indefinitely (Thomas et al., Science 282: 1145-1147 (1998)), offer a plentiful source of endothelial cells (ECs) for therapeutic revascularization. However, few studies have identified specific developmental stimuli sufficient to support the specification and maintenance of large numbers of functional and vascular-committed ECs from hESCs (Yamahara et al., PLoS ONE 3: e1666 (2008); Sone et al., Arterioscler. Thromb. Vasc. Biol. 27: 2127-2134 (2007); LU et al. Nat. Methods 4: 501-509 (2007); Goldman et al., Stem Cells 27: 1750-2759 (2009); Nourse et al., Arterioscler. Thromb. Vasc. Biol. 30: 80-89 (2009); Bai et al., J. Cell Biochem. 109(2):363-74 (2010), published online Nov. 30, 2009). Indeed, although few hESC-derived ECs have been generated in short-term cultures, these cells have not been subjected to sustained expansion, angiogenic profiling or interrogated as to the stability of vascular fate. As a result, the molecular pathways that maintain vascular identity and long-term expansion of hESC-derived ECs remain unknown. A major impediment to studies of EC differentiation from hESCs has been the lack of cell intrinsic genetic labeling tools for EC-specific lineage tracing.

### SUMMARY OF THE DISCLOSURE

This disclosure relates to generation of human endothelial cells (ECs) from human embryonic stem cells (ESCs) in culture. It has been recognized herein that inhibition of TGFβ signaling after mesoderm induction and during vascular differentiation of human embryonic stem cells (hESCs)-derived cells substantially enriches endothelial cells (ECs) in the cell population; and following isolation of these ECs, continued culturing of the isolated ECs in the presence of a TGFβ signaling inhibitor maintains the proliferative ability and phenotypic homogeneity of the ECs for extended culture periods.

In one aspect, the disclosure is directed to a method for developing human ECs from human ESCs in culture based on inhibition of TGFβ signaling after mesoderm induction and during vascular differentiation.

In specific embodiments, the method includes culturing human ESCs to form embryoid bodies (EBs); culturing EBs under conditions that induce and promote mesoderm specification; further culturing the cells under conditions that promote vascular differentiation thereby generating ECs; and further culturing the cells in the presence of a TGFβ signaling inhibitor, thereby expanding ECs in the cell population.

In one embodiment, EBs are cultured in the presence of an activin, a BMP and FGF-2 for a time sufficient for mesoderm induction. In a specific embodiment, the activin is activin A, the BMP is BMP4, and EBs are cultured for 4-6 days with the growth factors added at appropriate time to the culture media.

In another embodiment, following mesoderm induction, the cells are plated on an adherent substrate and cultured in media supplemented with VEGF-A, a BMP and FGF-2 to induce vascular differentiation. In a specific embodiment, the cells are cultured for 3-4 days.

In some embodiments, following induction of vascular differentiation, the cells are cultured in media supplemented with VEGF-A, FGF-2 and a TGFβ signaling inhibitor for at least 4-5 days, preferably at least 5-7 days, to sufficiently expand ECs in the cell population. ECs can be isolated from the cell population, and further cultured in the presence of a TGFβ signaling inhibitor if desired.

In specific embodiments, the TGFβ signaling inhibitor is an inhibitor specific for the type I TGFβ receptors. In some embodiments, the inhibitor is an inhibitor of ALK4, ALK5, and ALK7. In other embodiments, the inhibitor is an inhibitor of at least ALK5.

In one embodiment, the TGFβ signaling inhibitor is a soluble form of a type I receptor, an antibody directed to a type I receptor, or a small molecule compound. In specific embodiments, the inhibitor is a small compound selected from SB-431542, A 83-01, D 4476, LY 364947, SB 525334, SD 208, and SJN 2511.

In another aspect, this disclosure is directed to a substantially pure population of ECs. The ECs are characterized by expression of surface markers, VE-cadherin, CD31 and VEGFR2, and can proliferate and pass for extended culture periods without losing the characteristics of ECs.

In a further aspect, the instant disclosure provides a composition containing hESC-derived ECs, for example, a pharmaceutical composition that also includes one or more pharmaceutically acceptable carriers and diluents.

In still another aspect, this disclosure provides a method for repairing injured tissue in a human subject based on administering to the subject a composition containing the ECs disclosed herein to promote vascularization.

In a further aspect, the disclosure provides a hESC cell line stably transfected with a nucleic acid molecule which encodes a fluorescent protein, operably linked to the promoter region of the VE-cadherin gene. Such reporter cell line is useful for monitoring the development of ECs from hESCs.

### BRIEF DESCRIPTION OF THE DRAWINGS

FIG. 1A-G. Sequential TGFβ activation followed by inhibition during phase 1 differentiation promotes a tenfold expansion of hVPr-GFP+ hESC-derived cells. (A) A 1.5-kb fragment of the putative human VE-cadherin promoter (hVPr) region was isolated from a BAC clone and placed upstream of GFP in a lentiviral expression vector (hVPr-GFP). (B) Spontaneously differentiating embryoid bodies exhibited expression of hVPr-GFP in tubular structures. Inset, merge of GFP and brightfield views. (C) Flow cytometric analysis showed hVPr-GFP+ cells were positive for the vascular markers CD31 and VEGFR2. (D) Schematic diagram showing the sequence in which BMP4, activinA, FGF-2, VEGF-A and SB431542 were added and removed from differentiation cultures. EC, endothelial cell. (E,F) The proportion of hVPr-GFP+ cells was measured by flow cytometry at day 14 after culture in the absence (−SB; E) and presence (+SB; F) of SB431542. (G) Measurement of hVPr-GFP+ cells at day 14 when embryoid bodies were cultured either in groups or as isolated embryoid bodies and SB431542 was added at day 0, day 7 or not at all (N). Error bars represent s.d. of experimental values performed in triplicate. Scale bars, 100 μm.

FIG. 2A-J. TGFβ inhibition after endothelial cell isolation during phase 2 increases yield and preserves vascular identity of purified endothelial cells. (A-C) Human VPr-GFP hESCs were sequentially stimulated with cytokines (−SB) and SB431542 (+SB) (FIG. 1D) and cultures were assessed for the prevalence of pluripotency (Oct3/4) and mesodermal transcripts (brachyury): (A) CD31 and α-SMA transcripts, (B) endothelial cell markers hVPr-GFP and CD31, and (C) at multiple time points during differentiation. The secondary axis in B shows values for cells shown in solid bars. (D) Isolated endothelial cells that were cultured in the absence of SB431542 were stained for both VE-cadherin and α-SMA and showed rare cells that were positive for both markers (arrowhead in the inset). Inset, α-SMA alone. (E-I) Human VPr-GFP+ cells were isolated from differentiation cultures at day 14 by FACS and further cultured in the absence (E) or presence (F) of SB431542. (G) Flow cytometric assessment of CD31 was performed after 5 d of isolated culture (total cells are shown in white and CD31+ cells are shown in black in the bar graph). (H) After isolation and 5 d of culture in the presence or absence of SB431542, the incidence of α-SMA+ cells was measured. (I) After 5 d of culture following isolation, unstimulated cultures showed reduced incidence of cells positive for phospho-histoneH3 (PHH3+), relative to SB431542-stimulated cultures. The mean incidences of α-SMA and phospho-histoneH3 positive cells were obtained by counting positively stained cells in multiple parallel wells. (J) The yield of endothelial cells (ECs) from hESCs is schematized relative to a 50,000 hESC input at day 0. The relative difference in endothelial cell (ECs) number is indicated at day 14 (upon isolation from differentiation cultures), and day 20 (after expansion in isolated conditions). The ratio of input hESCs to committed hESC-derived endothelial cells after 20 d is also shown. Relative transcript abundance was measured by QPCR and normalized to the housekeeping gene β-actin (ACTB). Error bars in (A-C and G-I) represent s.d. of experimental values performed in triplicate. Scale bars, 100 μm.

FIG. 3A-C. Molecular profiling of hESC-derived endothelial cells reveals a signature defined by high Id1 expression. Human VPr-GFP embryoid bodies and highly purified hVPr-GFP+ cells were compared to mature vascular cells by microarray analysis. RNA was extracted for microarray analysis from human VPr-GFP embryoid bodies cultured in the presence of recombinant cytokines alone until day 14; isolated endothelial cells (99.8% pure) from hVPr-GFP embryoid bodies cultured in the presence of recombinant cytokines and the TGFβ inhibitor SB431542 until day 14; isolated endothelial cells (>95% pure) from hVPr-GFP embryoid bodies cultured in the presence of recombinant cytokines and the TGFβ inhibitor SB431542 until day 14, followed by 10 d additional culture in the presence of cytokines and SB431542; HUVECs; human umbilical vein smooth muscle cells; and CD34+ umbilical cord blood cells. (A) Following the endothelial cell differentiation protocol (FIG. 1D), Id1-YFP hESC-derived cells were sorted by FACS, separating the CD31+ population into Id1-YFPhigh-expressing cells and Id1-YFPlow-expressing cells. (B) After 3 d culture in the presence of SB431542, both populations were transferred to conditions with and without SB431542 for an additional 4 d (+SB and −SB, respectively). (C) Total cells and mean fluorescence intensity (MFI) measurements of Id1:YFP (black) and CD31+ (white) were measured for: CD31+Id1low (I) and CD31+Id1high (II) populations upon isolation; and for four populations following culture conditions (as shown in c, III-VI). Scale bars, 100 μM.

FIG. 4A-D. TGFβ inhibition upregulates Id1 expression and is necessary for the increased yield of functional endothelial cells capable of in vivo neoangiogenesis. (A,B) Human VPr-GFP hESCs that were stably transduced with control (A) or Id1-specific (B) shRNAs were differentiated according to the protocol shown in FIG. 1D and assessed at day 14 for the prevalence of VEGFR2+ (blue) and hVPr-GFP+ (green) cells. The insets show plots of side scatter on the y axis and hVPr-GFP on the x axis. (C) Control and Id1-specific shRNAs were added to HUVEC or freshly isolated (at day 14) hVPr-GFP+ cells, and the relative Id1 transcript levels were measured after 3 d. *, P<0.05. Error bars, s.d. of experimental values performed in triplicate. (D) Control and Id1-specific shRNAs were added to freshly isolated hVPr-GFP+ cells, which were cultured in the absence or presence of SB431542. After 5 d, the total cell number and proportion of CD31+ cells was measured by flow cytometry. Error bars, s.d. of experimental values performed in triplicate. Scr, scrambled control shRNA.

FIG. 5. Structure of the TGFβ inhibitor SB-431542 {4-(5-benzo[1,3]dioxol-5-yl-4-pyridin-2-yl-1H-imidazol-2-yl)-benzamide}.

### DETAILED DESCRIPTION

It has been recognized herein that inhibition of TGFβ signaling after mesoderm induction and during vascular differentiation of human embryonic stem cells (hESCs)-derived cells significantly enhances emergence of endothelial cells (ECs); and that following isolation of these ECs, TGFβ signaling inhibition preserves a high degree of proliferation as well as the phenotypic homogeneity of these ECs. Accordingly, this disclosure provides a method for developing and expanding human ECs from hESCs, ECs developed by this method, and therapeutic use of such ECs. A reporter hESC line useful for tracking the development of ECs is also provided.

Method of Developing and Expanding Human ECs

In one aspect, the disclosure is directed to a method for developing human ECs from human ESCs in culture based on inhibition of TGFβ signaling.

Without being limited to any particular theory, it is believed that inhibition of TGFβ signaling at an appropriate time during vasculogenic differentiation of human ESCs-derived cells in culture can selectively enrich endothelial cells in the cell population. Inhibition of TGFβ signaling is executed following mesoderm induction and when vascular differentiation has been initiated and at least some cells bearing characteristics of ECs have appeared in a hESC-derived cell population. Inhibition of TGFβ signaling at this point is believed to selectively promote the survival and expansion of ECs, as relative to non-endothelial cells. Premature inhibition of TGF signaling during the early stage of differentiation, however, would not permit generation of sufficient ECs because mesoderm induction from hESCs is dependent on TGFβ signaling.

Generally speaking, in accordance with the instant method, human ESCs are cultured under conditions that allow formation of embryoid bodies (EBs). Afterwards, EBs are cultured under conditions that induce and promote mesoderm specification, for example, suspension culture conditions in media supplemented with mesoderm promoting factors. Subsequently, the cells are cultured under conditions that promote vascular differentiation and generation of ECs. The cells are then exposed to a molecule that inhibits TGFβ signaling to expand ECs in the cell population. ECs can be subsequently purified from the cell population and further cultured in the presence of the TGFβ inhibitor if desired.

Formation of EBs

EBs are three dimensional aggregates of cells derived from ESCs, and contain a large variety of differentiating cell types or lineages.

Human ESCs can be obtained by methods known in the art. For example, human ESCs can be prepared from the inner cell mass (ICM) of blastocysts as described in, e.g., U.S. Pat. No. 5,843,780 to Thomson et al. or in Reubinoff et al. (Nature Biotech 18: 399, 2000). Alternatively, human ESCs may be obtained from commercial sources. Human ESCs can be cultured under self-renewal culture conditions, i.e., conditions that maintain pluripotency and ability to replicate of hESCs. Such conditions have been well documented in the art. Self-renewal conditions include both feeder-based conditions (e.g., mouse embryonic fibroblast as a feeder layer), and feeder-free conditions where the media is conditioned by feeder cells. Both serum-containing media and defined, serum-replacement media can be used. Certain growth factors have also been identified to support self renewal of hESCs, such as FGF-2. Culture media that support self-renewal of hESCs are also available from various commercial sources.

In specific embodiments, human ESCs are initially maintained under feeder-free conditions on a substrate covered with a membrane, e.g., Matrigel™ (BD Biosciences), in serum-free defined media conditioned by mouse embryonic fibroblast (MEF), in the presence of FGF-2 (e.g., 4 ng/ml). While feeder cells and feeder-conditioned media are believed to promote self renewal and inhibit differentiation of hESCs, neither is used in subsequent steps.

To induce formation of EBs, human ESCs cultured under self-renewal conditions may be treated to precondition the cells for formation of EBs. Such preconditioning can include, e.g., removal of FGF-2 from the media, and addition of a bone morphogenetic protein (BMP) at a low concentration (e.g., 2 ng/ml BMP4, optionally in combination with BMP2). Human ESCs can be cultured in such pre-conditioning media for about 1-2 days.

Formation of EBs from human ESCs can be achieved using methods known in the art. For example, human ESCs maintained under self renewal conditions, which in some embodiments have been preconditioned, are dissociated from the substrate, resuspended and cultured undisturbed in media devoid of FGF2 and supplemented with a BMP for 1-2 days, for example, for about 18-24 hours, to form EBs. Typically, the plates used at this stage are low attachment plates in order to keep cells in suspension and facilitate formation of EBs. In specific embodiments, the BMP is BMP4 and is used at a concentration in the range of 2-5 ng/ml, or about 2.5 ng/ml, optionally in combination with BMP2 at the same concentration.

EBs can also be formed from induced pluripotent stem (iPS) cells. iPS cells refer to a type of pluripotent stem cells artificially derived from a non-pluripotent cell, typically an adult somatic cell, by inducing reprogrammed expression of specific genes. A non-pluripotent cell can be induced to become a pluripotent cell by genetic modification (e.g., transfection of certain stem-cell associated genes), or by proteins (e.g., repeated treatment with proteins channeled into the cells through poly-arginine anchors), among other means. Formation of EBs from iPS cells has been described, for example, by Takahashi et al. (Cell 131:861, (2007)) and Mali et al. (Stem Cells 26:1998 (2008)).

Culturing EBs to Induce Mesoderm Specification

After formation of EBs, EBs are fed with media that promote mesoderm induction and specification. More specifically, EBs are initially cultured in a medium supplemented with an activin and a BMP. In specific embodiments, the activin is activin A, the BMP is BMP4 or a combination of BMP4 and BMP2, and EBs are left undisturbed in media containing activin A and BMP4 for about 1-2 days. Subsequently, FGF-2 is added to the culture medium, i.e., the cells are cultured in media containing an activin, a BMP and FGF-2, and the culture is continued for additional 2-3 days.

The concentration of activin A is generally from about 1.0 ng/mL to about 30 ng/mL. In some embodiments, the concentration of activin A is from about 5 ng/mL to about 15 ng/mL. In a specific embodiment, the activin A is used at about 10.0 ng/mL.

The concentration of BMP4 is generally from about 1.0 ng/mL to about 30 ng/mL. In some embodiments, the concentration of BMP4 is from about 10 ng/mL to about 25 ng/mL. In a specific embodiment, the concentration of BMP4 is about 20 ng/mL.

The concentration of FGF-2 is generally from about 1.0 ng/mL to about 30 ng/mL. In some embodiments, the concentration of FGF-2 is from about 5 ng/mL to about 15 ng/mL. In a specific embodiment, the concentration of FGF-2 is about 8 ng/mL.

In a specific embodiment, upon formation, EBs are cultured for about 1 day in the presence of 10 ng/mL of activin A and 20 ng/mL of BMP4; then 8 ng/mL FGF-2 is added to the media, and the cells are cultured for additional 2 days.

Vascular Differentiation on an Adherent Substrate

After mesoderm induction, the cells are harvested and transferred to an adherent substrate, and cultured under conditions that promote vascular differentiation.

Adherent substrates suitable for use herein are not limited to any specific type and include any substrate that permits cells to attach and grow in monolayers, such as tissue culture plates coated with gelatin, or coated with an extracellular matrix protein such as fibronectin, laminin or those contained in a Matrigel™ membrane. In a specific embodiment, growth factor reduced Matrigel™-coated tissue culture plates are used.

The culture media is supplemented with growth factors that promote vascular differentiation, for example, VEGF-A, FGF-2 and a BMP (e.g., BMP4), and no longer contains activin.

The concentration of VEGF-A is generally from about 5 ng/mL to about 40 ng/mL. In some embodiments, the concentration of VEGF-A is from about 15 ng/mL to about 30 ng/mL. In a specific embodiment, the concentration of VEGF-A is about 25 ng/mL.

The concentrations of BMP4 and FGF-2 are the same as described above for mesoderm induction.

The cells are cultured on an adherent substrate in media supplemented with VEGF-A, FGF-2 and a BMP for a period of time until at least some cells bearing characteristics of endothelial cells appear in the cell population. Generally speaking, the cells are cultured for about 3-4 days.

In a specific embodiment, after mesoderm induction, the cells are cultured on Matrigel™-coated plates for about 3 days, typically undisturbed, in media supplemented with 25 ng/mL VEGF-A, 8 ng/mL FGF-2, and a 20 ng/mL BMP4.

Inhibition of TGFβ Signaling to Expand ECs

After the cells have been cultured under conditions that induce vascular differentiation as described above, cells bearing characteristics of endothelial cells emerge in the culture. Addition of a TGFβ signaling inhibitor to the cell culture from this point on greatly enriches endothelial cells in the cell population by increasing both the percentage and absolute number of ECs.

In some embodiments, the cells are cultured on an adherent substrate in media containing a TGFβ signaling inhibitor, VEGF-A and FGF-2, and without BMP. VEGF-A and FGF-2 are factors that support cells of the vascular lineage and used at the same concentrations as described above for vascular differentiation. The cells are cultured for a time sufficient to enrich the ECs in the population, generally for at least 4-5 days, and in specific embodiments, for at least 5-7 days, and in other embodiments for a period of time longer than 7 days.

TGFβ signaling inhibitors suitable for use in the present method include any molecules that inhibit the activin/nodal branch of TGFβ superfamily signaling.

TGFβ superfamily signaling is mediated by two classes of receptors, the type I or activin like kinase (ALK) receptors, and type II receptors. Type I receptors include ALK4 (type I receptor for activin or inhibin), ALK5 (type I receptor for TGFβ) and ALK7 (type I receptor for nodal).

In certain embodiments, TGFβ signaling inhibitors used herein are selective inhibitors of type I receptors, i.e., inhibitors having differential (i.e., selectivity) for type I receptors relative to type II receptors. Selectivity can be measured in standard assays as an IC₅₀ ratio of inhibition in each assay. The inhibitor can be a specific inhibitor of one type I receptor (i.e., one of ALK4, ALK5 or ALK7), or an inhibitor that inhibits signaling of several type I receptors (e.g., all of ALK4, ALK5 and ALK7).

In a specific embodiment, the inhibitor inhibits at least ALK5-mediated signaling. ALK5, upon activation, phosphorylates the cytoplasmic proteins smad2 and smad3. The phosphorylated smad proteins translocate into the nucleus and activate certain gene expression. Inhibitors of ALK5-mediated signaling can be compounds that inhibit the kinase activity of ALK5 and block phosphorylation of smad proteins. See, e.g., review by Yingling et al., Nature Reviews (Drug Discovery) 3: 1011-1022 (2004).

The inhibitors can be polypeptides, such as soluble forms of TGFβ receptors (e.g., polypeptides composed of the extracellular segment of a receptor), particularly soluble forms of type I receptors, or antibodies directed to a TGFβ receptor particularly a type I receptor.

The inhibitors can be small molecule compounds as well. By “small molecule compounds” it is meant small organic compounds, generally having a molecule weight of less than 800 daltons. Small molecule inhibitors of TGFβ signaling have been well-documented in the art, including pyridyl substituted triarylimidazoles disclosed in U.S. Pat. No. 6,465,493 and US 20030149277 A1, pyridyl substituted imidazoles disclosed in US 20030166633 A1 and US 20040220230 A1, pyridyl substituted triazoles disclosed in US 20040152738 A1, thiazolyl substituted triazoles disclosed in US 20040266842 A1, 2-amino-4-(pyridin-2-yl)-thiazole derivatives disclosed in US 20040063745 A1, 2-pyridyl substituted diarylimidazoles disclosed in US 20040039198 A1, phenyl substituted triazoles disclosed in US 20050014938 A1, benzoxazine and benzoxazinone substituted triazoles in US 20050165011 A1 isoquinoline derivatives disclosed in US 20070072901 A1, thiazolylimidazole derivatives disclosed in US 20070154428 A 1, heteroaromatic compounds substituted with at least one 2-pyridyl moiety disclosed in U.S. Pat. No. 7,417,041, as well as those reviewed by Yingling et al., Nature Reviews (Drug Discovery) 3: 1011-1022 (2004), the contents of all of these publications are incorporated herein by reference. Small molecule inhibitors are also available through various commercial sources. For example, compounds listed in the following table are available through Tocris Bioscience (Missouri, USA), and are suitable inhibitors for use in the present methods. Additional small molecule inhibitors are available through EMD4Bisciences (New Jersey, USA).

| Compound | Chemical Name/Function |
|---|---|
| A 83-01 | 3-(6-Methyl-2-pyridinyl)-N-phenyl-4-(4-quinolinyl)-1H- |
| | pyrazole-1-carbothioamide |
| | (Selective inhibitor of ALK5, ALK4 and ALK7) |
| D 4476 | 4-[4-(2,3-Dihydro-1,4-benzodioxin-6-yl)-5-(2-pyridinyl)- |
| | 1H-imidazol-2-yl]benzamide |
| | (Selective CK1 inhibitor. Also inhibits ALK5) |
| LY 364947 | 4-[3-(2-Pyridinyl)-1H-pyrazol-4-yl]-quinoline |
| | (Selective inhibitor of ALK5) |
| SB 431542 | 4-[4-(1,3-benzodioxol-5-yl)-5-(2-pyridinyl)-1H-imidazol-2- |
| | yl]benzamide |
| | (selective inhibitor of ALK5, ALK4 and ALK7) |
| SB 525334 | 6-[2-(1,1-Dimethylethyl)-5-(6-methyl-2-pyridinyl)-1H- |
| | imidazol-4-yl]quinoxaline |
| | (Selective inhibitor of ALK5) |
| SD 208 | 2-(5-Chloro-2-fluorophenyl)-4-[(4-pyridyl)amino]pteridine |
| | (Potent ATP-competitive ALK5) |
| SJN 2511 | 2-(3-(6-Methylpyridine-2-yl)-1H-pyrazol-4-yl)-1,5- |
| | naphthyridine |
| | (Selective inhibitor of ALK5) |

In one embodiment, the compound, SB-431542, is used as a TGFβ signaling inhibitor. This compound is added to the culture media at a concentration ranging from about 1 μM to about 15 μM, or about 2 μM to about 10 μM. In a specific embodiment, this compound is added to the media at about 10 μM. Appropriate concentrations for other small molecule inhibitors may depend on the structure or functional mechanism of a particular inhibitor and may be in the micromolar range, which can be determined by those skilled in the art (e.g., based on IC50 values determined in appropriate in vitro assays).

Emergence of ECs in the culture can be determined based on growth characteristics, morphological features, cell surface phenotypes, transcription profiles, or a combination of any of these characteristics. For example, ECs grow as monolayers when cultured on adherent substrates, and divide about every 24-36 hours. Morphologically, ECs are about 10 μm in length, and of a “fried-egg” or cobblestone shape. Cell surface markers characteristic of ECs include VE-cadherin+, VEGFR2+, and CD31+. At the level of transcription, human ECs are characterized by expression of VE-cadherin, VEGFR2, Id1, Thrombomodulin, and EphrinB2.

hESC-derived ECs disclosed herein are also distinguished from mature ECs such as human umbilical vein endothelial cells (HUVECs). While both hESC-derived ECs and mature ECs are positive for expression of cell surface markers VE-cadherin, VEGFR2 and CD31, hESC-derived ECs may express α-SMA, which is not expressed in mature ECs. Further, the transcription profile of hESC-derived ECs can be defined by a VE-cadherin⁺VEGFR2^{high}Id1^{high} Thrombomodulin^{high}Ephrin B2⁺CD133⁺HoxA9⁻ phenotype, while mature ECs can be identified as VE-cadherin⁺VEGFR2^{low}Id1^{low}EphrinB2⁺CD133⁻HoxA9⁺.

As a result of culturing in the presence of a TGFβ signaling inhibitor, the ECs in the cell population are substantially enriched. By “substantially enriched” it is meant that the percentage of ECs in a cell population has been increased by at least 1 fold (100%), 2 fold, 3 fold, 4 fold, 5 fold, 6 fold, 7 fold, 8 fold, 9 fold, 10 fold, or greater.

Isolation of ECs and Substantially Pure, Stable ECs

After enrichment, ECs can be isolated from the cultured cell population to provide a substantially pure and stable population of ECs. By “substantially pure” it is meant that ECs account for at least 75%, 80%, 85%, 90%, 95%, 98%, 99% or greater percentage of the cells in the cell population. By “stable” it is meant that ECs can be cultured for extended period of time, e.g., at least 5 passages, at least 10 passages, at least 15 passages or longer, without losing the characteristics of ECs.

Isolation of ECs can be achieved using antibodies specific for EC surface markers, such as VE-cadherin, CD31 or VEGFR2, attached to magnetic beads or fluorophores for use in Magnetic or Fluorescence Activated Cell Sorting (MACS or FACS).

Isolated ECs can continue to be cultured in media supplemented with VEGF and FGF-2 in the presence of a TGFβ inhibitor. The use of a TGFβ inhibitor at this stage has been shown herein to further promote the growth and expansion of ECs without losing the surface phenotype characteristic of ECs for an extended culture period, for example, for at least 10 passages.

As shown hereinbelow, isolated hESC-derived ECs are capable of further differentiating into vessels in vivo.

Pharmaceutical Compositions and Therapeutic Methods

The culture method disclosed herein permits a reproducible production of large numbers of stable human ECs, which are useful for therapeutic vascularization of injured tissues.

Accordingly, in a further aspect, the instant disclosure provides a composition containing hESC-derived ECs. The composition can include one or more pharmaceutically acceptable carriers and diluents. The composition can also include components that facilitate engraftment.

In a further aspect, this disclosure is directed to therapeutic uses of the endothelial cells provided herein. For example, the instant endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the instant endothelial cells can be further modified to deliver agents to target and treat tumors.

In specific embodiments, this disclosure provides a method of repair or replacement for tissue in need of vascular cells or vascularization. This method involves administering to a human subject in need of such treatment, a composition containing the isolated ECs to promote vascularization in such tissue.

The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

Promoting angiogenesis in a tissue can be beneficial to individuals who have or are at risk to develop a condition including an ischemic condition, e.g., myocardial infarction, congestive heart failure, and peripheral vascular obstructive disease, stroke, reperfusion injury, limb ischemia; neuropathy (e.g., peripheral neuropathy, or diabetic neuropathy), organ failure (e.g., liver failure, kidney failure, and the like), diabetes, rheumatoid arthritis, and osteoporosis.

The present endothelial cells or a composition containing such cells can be administered in a manner that results in delivery or migration to or near the issue in need of repair or vascularization. In some embodiments, the cells are systemically administered and circulate to the tissue in need thereof; or alternatively, locally administered, e.g., delivered directly (by injection, implantation or any suitable means) into the tissue or nearby tissue which is in need of these cells. In other embodiments, the cells are integrated into an artificially engineered tissue prior to implantation.

In another embodiment, this disclosure provides a method of targeting certain agents to tumors in a subject by administering to the subject the endothelial cells that have been engineered for delivery of such agents. Because tumors frequently stimulate the in-growth of new blood vessels into the tumor (stimulate tumor angiogenesis), endothelial cells delivered to a subject can contribute to the new tumor vasculature. Thus, the cells can be used to deliver agents directly to a tumor site. Examples of agents that can be targeted to tumors using endothelial cells include, but are not limited to, cytotoxic drugs, other toxins, radionuclides, and gene expression products. For example, endothelial cells can be engineered such that they also express a protein having anti-tumor activity, or such that they secrete, release, or are coated with a toxic agent such as a chemotherapeutic agent or radionuclide. For example, radionuclide drugs or chemotherapeutic drugs can be conjugated to an antibody that binds to the surface of the endothelial cells and thereby used to deliver the radionuclides or chemotherapeutic drugs to a tumor.

A hESC Reporter Line

Another embodiment of this disclosure is directed to a hESC cell line stably transfected with a nucleic acid molecule which encodes a fluorescent protein, operably linked to the promoter region of the VE-cadherin gene, also referred to herein as Vpr-GFP hESC reporter line. Since VE-cadherin is specifically expressed primarily in endothelial cells, the fluorescent protein is only expressed in cells that have differentiated into ECs from hESCs. Hence these cells are useful in screening for substances which induce this differentiation and for tracking of ECs. These cells are also useful in the isolation of ECs by FACS.

Fluorescent proteins suitable for use in making a reporter line includes such as green fluorescent protein (GFP), blue fluorescent protein (BFP), mOrange fluorescent protein, mCherry fluorescent protein, and yellow fluorescent protein (YFP).

This hESC reporter line is developed by introducing into hESCs a vector containing a nucleic acid molecule coding for a fluorescent protein, placed under the control of the VE-cadherin promoter. The vector can be introduced by any suitable method, such as by transfection or by viral-mediated transduction. In one embodiment, the vector is a lentivural vector, and lentivirus-mediated transduction is used to introduce the vector into hESCs. Transduced hESCs are screened to identify clones in which the vector has been stably integrated in the host genome. Cell lines are then established from the identified clones that are capable of self-renewal, have normal karyotype, have normal differentiation capability, and exhibit faithful and robust expression of the reporter in endothelial cells.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Example 1

### General Methods

Human ESC Culture

The experiments delineated here were performed primarily with the recently approved RUES1 hESC (kindly provided by Dr. Ali Brivanlou (James et al., Dev. Biol. 295: 90-102 (2006)) and corroborated using WMC2, WMC7, WMC8, which were hESC lines generated at Weill Cornell Medical College (kindly provided by Dr. Zev Rosenwaks/Dr. Nikica Zaminovic), H9 (Id1-YFP, kindly provided by Dr. Robert Benezra/Hyungsong Nam and Dr. Lorenz Studer/Dr. Mark Tomishima), and IPSc (kindly provided by Dr. Studer/Dr. Gabsang Lee). Human ESC culture medium consisted of Advanced DMEM/F12 (Gibco) supplemented with 20% Knockout Serum Replacement (Invitrogen), 1× ential amino acids (Gibco), 1× L-Glutamine (Invitrogen), 1× Pen/Strep (Invitrogen), 1× βMercaptoethanol (Gibco), and 4 ng/ml FGF-2 (Invitrogen). Human ESCs were maintained on Matrigel™ using hESC medium conditioned by mouse embryonic fibroblasts (MEF, Chemicon).

Embryoid Bodies

Human VPr-GFP hESCs were grown to confluence on Matrigel™ (BD Bioscience) and then incubated in 5 units/ml dispase (Gibco) until colonies were completely detached from the substrate. Human VPr-GFP EBs were washed and cultured in hESC medium on ultra low attachment plates (Corning) and cultured in the conditions described, with replacement of cytokine supplemented medium every 48 hours. Embryoid bodies were fixed in 4% paraformaldehyde and frozen for cryosectioning and staining.

Endothelial (EC) Differentiation

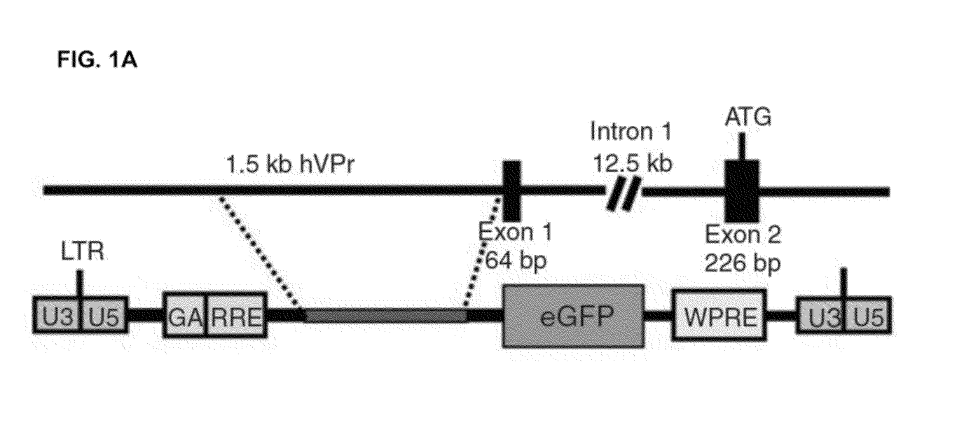

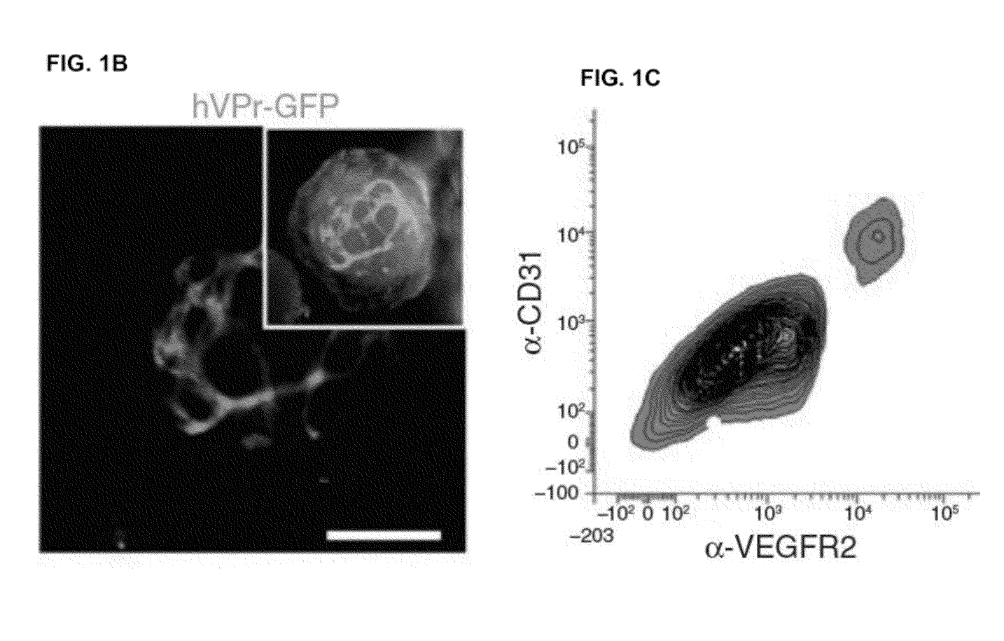

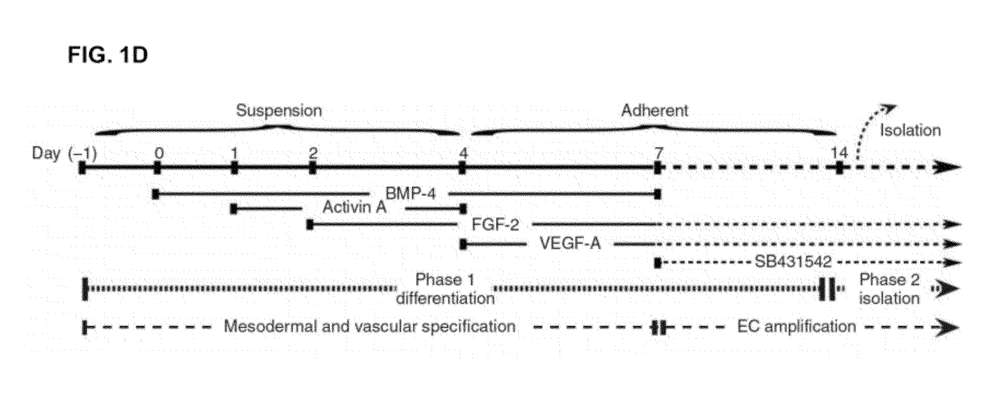

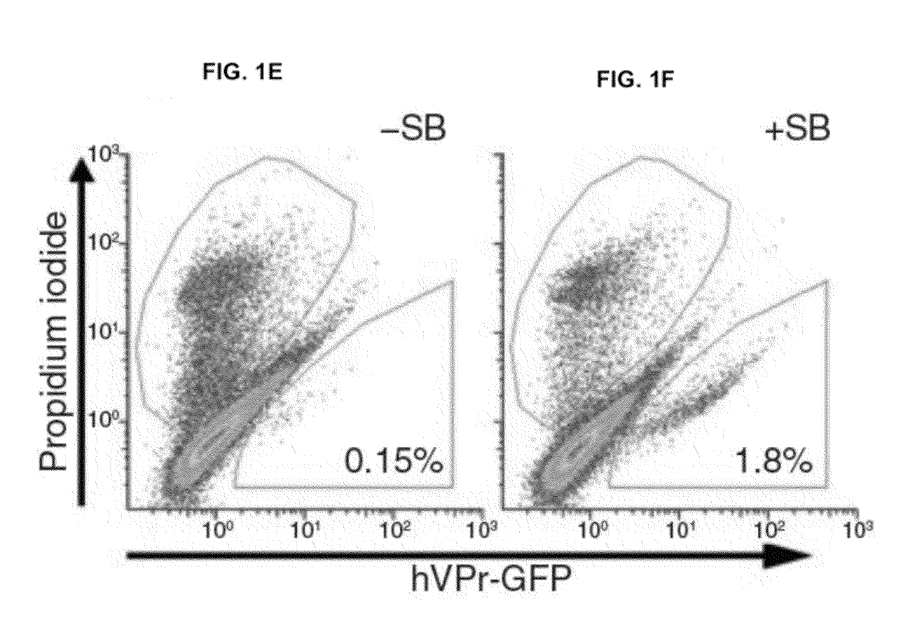

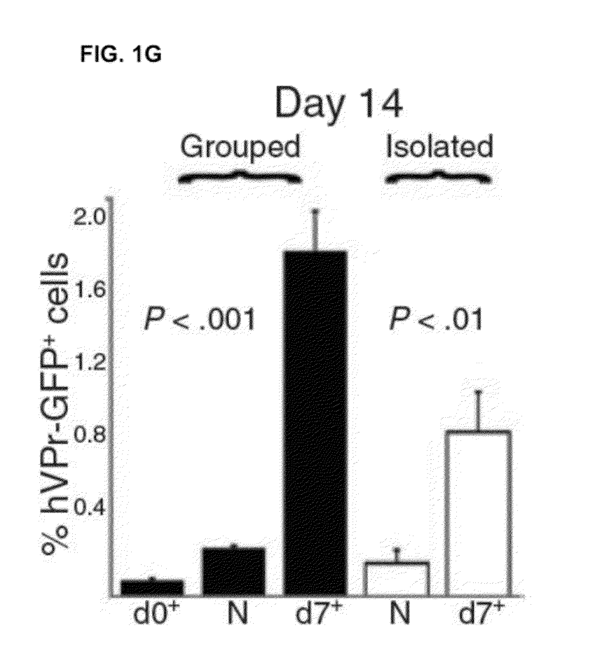

Embryoid bodies were generated and cultured in base hESC medium, supplemented with the cytokines as shown. Sequential administration of cytokines was implemented as shown in FIG. 1D. Briefly, embryoid bodies were generated in hESC base medium without FGF-2. On the morning following generation of EBs (day 0), medium was supplemented with 20 ng/ml BMP4 (R&D) (removed at day 7); on day 1, medium was supplemented with 10 ng/ml ActivinA (R&D) (removed at day 4); on day 2, medium was supplemented with 8 ng/ml FGF-2 (Peprotech) (remained for the duration of culture); on day 4, EBs were transferred to adherent conditions on Matrigel™-coated plates and medium was supplemented with 25 ng/ml VEGF-A (Peprotech) (remained for the duration of culture); on day 7, SB431542 (Tocris) was added at 10 μM concentration and remained for indicated duration. Cultures were dissociated using 0.5% Trypsin/EDTA (Gibco) or Accutase (eBioscience). Absolute yield as well as ratio of input hESCs to differentiated ECs was calculated from the number of live cells recovered from differentiation cultures at days 0, 14 and 20. Purified ECs could be frozen and thawed in 10% DMSO with greater than 90% recovery.

Endothelial Cell Isolation and Flow Cytometry

ECs were isolated from differentiation cultures using Magnetic Activated Cell Sorting (MACS; Miltenyi Biotech) with an antibody against CD31 conjugated to magnetic microbeads. Alternatively, cells were isolated by virtue of the expression of GFP/YFP or a fluorophore conjugated antibody to human CD31 or VEGFR2 (BD) using a FACS AriaII (BD).

Quantitative PCR

Total RNA was prepared from cultured cells using the RNeasy™ extraction kit (Qiagen) and reverse transcribed using Superscript II reverse transcriptase (Invitrogen) according to the manufacturer's instructions. Relative quantitative PCR was performed on a 7500 Fast Real Time PCR System (Applied Biosystems) using either TaqMan PCR mix along with Id1 and β-actin primer pairs, or SYBR Green PCR mix (Applied Biosystems). Human specific SYBR green primer pairs used were: PECAM-f, 5′-tctatgacctcgccctccacaaa-3′ (SEQ ID NO: 1), r, 5′ gaacggtgtcttcaggttggtatttca-3′ (SEQ ID NO: 2); Oct3/4-f, 5′-aacctggagtttgtgccagggttt-3′(SEQ ID NO: 3), r, 5′-tgaacttcaccttccctccaacca-3′ (SEQ ID NO: 4); Brachyury-f, 5′-cagtggcagtctcaggttaagaagga-3′ (SEQ ID NO: 5), r, 5′-cgctactgcaggtgtgagcaa-3′ (SEQ ID NO: 6); and α-SMA, f, 5′-aatactctgtctggatcggtggct-3′ (SEQ ID NO: 7), r, 5′-acgagtcagagctttggctaggaa-3′ (SEQ ID NO: 8). Cycle conditions were: one cycle at 50° C. for 2 min followed by 1 cycle at 95° C. for 10 minutes followed by 40 cycles at 95° C. for 15 s and 60° C. for 1 minute. Primers were checked for amplification in the linear range and primer dissociation and verified. Threshold cycles of primer probes were normalized to the housekeeping gene β-actin (ACTB) and translated to relative values.

Microarray Analysis

The Affymetrix Human Genome U133 2.0 array was used to analyze gene expression. In brief, using Qiagen RNeasy™ kits, total RNA was extracted from: Human VPr-GFP EBs that were cultured in the presence of recombinant cytokines alone until day 14; MACS sorted ECs isolated from hVPr-GFP EBs cultured in the presence of recombinant cytokines alone until day 14; MACS sorted ECs isolated from hVPr-GFP transduced EBs cultured in the presence of recombinant cytokines and the TGFβ inhibitor SB431542 until day 14; MACS sorted ECs isolated from hVPr-GFP EBs cultured in the presence of recombinant cytokines and the TGFβ inhibitor SB431542 until day 14, followed by 10 days additional culture in the presence of cytokines and SB431542; human umbilical vein ECs; human umbilical vein smooth muscle cells; and CD34⁺ umbilical cord blood cells. The Superscript choice kit (Invitrogen, Carlsbad, Calif.) was used to make cDNA with a T7-(dT)24 primer incorporating a T7 RNA polymerase promoter. The biotin labeled cRNA was made by in vitro transcription (Enzo Diagnostics). Fragmented cRNA was hybridized to the gene chips, washed, and stained with streptavidin phycoerythrin. The probe arrays were scanned with the Genechip System confocal scanner and Affymetrix Microarray suite 4.0 as used to analyze the data.

Mouse In Vivo Test of Differentiated ECs in Matrigel™ Plug

Human VPr-GFP EBs were differentiated for 14 days by the differentiation protocol described above, followed by expansion in the presence of SB431542 for 10 days and injected subcutaneously into NOD/SCID mice in a suspension of Matrigel™. After 2 weeks, Griffonia simplificolia IB4 lectin and/or Ulex europus agglutinin lectin were administered intra-vitally to Matrigel™ plug bearing mice and plugs were harvested, fixed overnight in 4% paraformaldehyde and equilibrated in 30% sucrose before freezing and cryosectioning.

Immunofluorescence

Cryosections were immunocytochemically stained as previous described (James et al., Dev. Biol. 295: 90-102 (2006)). Briefly, samples were permeabilized in PBST and blocked in 5% donkey serum. Samples were incubated for 2 hours in primary antibodies blocking solution, washed 3 times in PBS and incubated in CY3-conjugated secondary antibodies (Jackson Laboratories) for 1 hour. Following washing some sections were counterstained for nucleic acids by TO-PRO3 (Invitrogen) before mounting and imaging by confocal microscopy. Primary antibodies included CD31 (DAKO), CD34 (DAKO), Phospho-HistoneH3, Smooth Muscle Actin (SMA) (DAKO) and VE-cadherin (R&D). All imaging was performed using a Zeiss 510 META confocal microscope.

Live Imaging and 3D Rendering

Human VPr-GFP EBs were cultured in a TOKAI-HIT™ live cell-imaging chamber on a Zeiss 510 META confocal microscope. Laser intensity and interval were optimized to ensure viability of cells for the duration of the experiments. Three dimensional reconstruction and rendering of optical z-stacks were performed using Improvision Volocity™ software.

### Example 2

### Generation of hESC Lines Expressing Green Fluorescent Protein Under Control of the Promoter for the Human VE-Cadherin Gene

To detect the emergence of ECs from differentiating hESCs in real-time, a cell line for EC-specific lineage tracing was generated. A 1.5 kilobase fragment (SEQ ID NO: 9) was isolated from a bacterial artificial chromosome (BAC) containing the human VE-cadherin genomic locus. The promoter sequence for this EC-specific gene, encompassing a region upstream of exon 1, was inserted into a lentiviral-vector upstream of GFP (hVPr-GFP) (FIG. 1A).

Ordinarily, if a constitutively expressed means of positive selection is absent from the vector, cells in which viral integration has occurred cannot be readily distinguished from non-transduced cells, as the tissue specific reporter contained within the lentiviral vector is expected to be expressed only in specific differentiated derivatives. A protocol that utilized lentiviral vectors without constitutively expressed reporters for positive selection was used herein. This protocol exploited a unique quality of the lentiviral vector (Follenzi et al., Nat Genet. 25: 217-222, 2000), which provided transient weak expression of the EC-specific reporter transgene following transduction, which was ultimately silenced in undifferentiated hESC derivatives. By isolating the subpopulation of cells that briefly expressed the reporter gene during this window (approximately two days after transduction), clonal derivatives in which viral integration took place were enriched.

Supernatants containing infectious lentiviral particles were collected 40 and 68 hours after transfection of HEK 293T with hVPr-GFP along with accessory vectors as previously described (Naldini et al., Science 272: 263-267 (1996)). Viral supernatants were concentrated by ultracentrifugation and used to transduce undifferentiated RUES1 hESCs. Essentially, concentrated lentivirus particles at relatively high multiplicity of infection (“MOI”) (between 5 and 10) were added to hESC colonies in MEF-conditioned medium. After 24 hours, the lentivirus-containing medium was replaced with fresh MEF-conditioned medium, and the cells were incubated for another 24 hours. The MEF-conditioned medium was then replaced with hESC base medium for a brief period of incubation (3 hours). Subsequently, hESCs were disaggregated by accutase to form single cells, which were sorted by FACS. Using non-transduced cells as a negative control, the population of hESCs that showed expression of the transgene was collected. The collected population of cells were plated on Matrigel™-coated plates, and cultured until substantial colonies emerge with morphological hallmarks of homogeneous self-renewal.

Colonies were examined for a few parameters: a) self-renewal, b) normal karyotype, c) normal differentiation capability, and d) faithful and robust expression of the reporter construct in endothelial cells. For criteria d), in order to determine whether the reporter was active, each of the candidate clones was divided into two cultures: one culture was cultured and expanded under self-renewing conditions, and the other was differentiated to ECs based on the protocol described in Example 1. Clones that show robust expression of the reporter gene were selected. Clones (or “lines”) that met all the above criteria were archived in liquid nitrogen and one specific clone/line was used in subsequent experiments.

Using the protocol described above, hESC clones (or “lines”) transduced with a reporter construct having the mOrange fluorescent protein as the reporter, were also generated and named VPr-mOrange hESC lines.

### Example 3

### Generation of an Id1 hESC Reporter Line

A bacterial artificial chromosome (BAC) was modified in order to place yellow fluorescent protein (YFP) under control of the endogenous human Id1 promoter locus. This reporter construct was electroporated into the H9 hESC line, selected for BAC integration using antibiotic resistance and subcloned. Clones were assessed and selected based on expression of YFP in Id1 hESC derivatives following spontaneous differentiation.

### Example 4

### Tracking Vasculogenic Differentiation Using the hVPr-GFP hESC Reporter Line

The hVPr-GFP hESC reporter line described in Example 2 above enabled the tracking of the chronology and geometry of vasculogenic differentiation using time-lapse confocal microscopy. When this reporter cell line was subjected to the EC differentiation protocol described in Example 1, commencing at day 5, the specification and emergence of hVPr-GFP⁺ ECs were observable, and by day 8, hVPr-GFP⁺ ECs co-expressing VEGFR2 and CD31 (FIG. 1B-C) formed motile microcapillary-like structures expressing EC markers, including VE-cadherin, CD31 and CD34, and were negative for alpha smooth muscle actin (α-SMA) and CD45, a marker for hematopoietic cells. When the hVPr-GFP lentiviral vector was used to transduce the non-endothelial cell types, human mesenchymal cells, foreskin fibroblastic cells and smooth muscle cells, GFP was not expressed. On the other hand, robust GFP expression was observed in human umbilical vein ECs (HUVECs) transduced with the hVPr-GFP construct. These data validated the ability of the hVPr-GFP reporter construct to specifically identify and track hESC-derived ECs.

This EC reporter hESC line was also used to monitor the development of a chemically defined, serum-free methodology that could effectively augment vascular differentiation, consisting of two phases. In phase 1, heterogeneous EB cultures of hVPr-GFP-hESCs were sequentially stimulated with bone morphogenetic protein (BMP) 4, ActivinA, fibroblast growth factor (FGF)-2, and VEGF-A (Huber et al., Nature 432: 625-630 (2004); Levenerg et al., Blood 110: 806-814 (2007); Yang et al., Nature 453: 524-528 (2008)) (FIG. 1D). Although these growth conditions promoted formation of hVPr-GFP⁺ structures, the yield of dissociated hESC-derived ECs obtained by fluorescence-activated cell sorting (FACS) was low, and the few isolated ECs could not be expanded without the majority of derivatives assuming a non-EC phenotype.

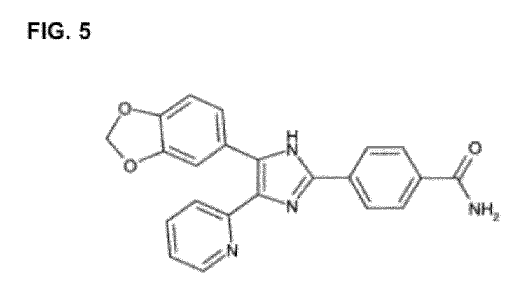

To generate more substantive yields of ECs, this hESC reporter cell line was screened for bioactive small molecules that enhanced differentiation of hESCs into hVPr-GFP+ ECs. After screening over 20 bioactive molecules (Table 1), it was determined that the TGF inhibitory molecule SB431542 (Inman et al., Mol. Pharmacol. 62: 65-72 (2002)) (FIG. 5) reproducibly elicited an increase in the yield of hVPr-GFP⁺ ECs. Adding SB431542 to differentiation cultures at day seven resulted in formation of hVPr-GFP⁺ VEcadherin⁺ monolayers, which upon dissociation, yielded ten-fold more ECs than cultures stimulated by cytokines alone (FIG. 1E-G). Notably, inclusion of SB431542 from the onset of differentiation (day 0) resulted in absence of hVPr-GFP+ ECs, indicating that vascular commitment is dependent on active TGFβ/Activin/Nodal signaling before day seven of differentiation.

TABLE 1
List of small bioactive molecules tested for their effect on
hVPr-GFP transduced hESCs.

6-promo-indirubin-3′-oxime (BIO) - GSK3β inhibitor
γ-secretase - Notch pathway inhibitor
AEG 3482 - Inhibitor of JNK signaling
AF 12198 - Selective type I IL-1 receptor antagonist
AG1478 hydrochloride - EGFR-kinase inhibitor
Anisomycin - Activator of JNK/SAPK/p38 MAP kinase
Celastrol - inhibitor of TNF-α-induced NF-κB activation
Cucurbitacin I - selective inhibitor of STAT signaling
Cyclopamine - Hedgehog signal inhibitor
Demethylasterriquinone B1 - Selective insulin RTK activator
Dorsomorphin hydrochloride - BMP type I receptor inhibitor
Fumagilin - Methionine aminopeptidease-2 inhibitor
Lestaurtinib - JAK2, FLT3 and TrkA inhibitor
LY 294002 - Selective PI3-Kinase inhibitor
PD173074 - Selective FGFR1 and FGFR3 inhibitor
Picropodophyllotoxin - Selective IGF1R inhibitor
Purmorphamine - Hedgehog signaling agonist
Rapamycin - mTOR inhibitor
SB203580 - Selective inhibitor of p38 MAPK
SB431542 - Selective inhibitor of TGF-βR1, ALK4, ALK5 and ALK7
SU5416 - VEGF receptor inhibitor
Thalidomide - TNF-α synthesis inhibitor
ZM 449829 - Selective JAK inhibitor

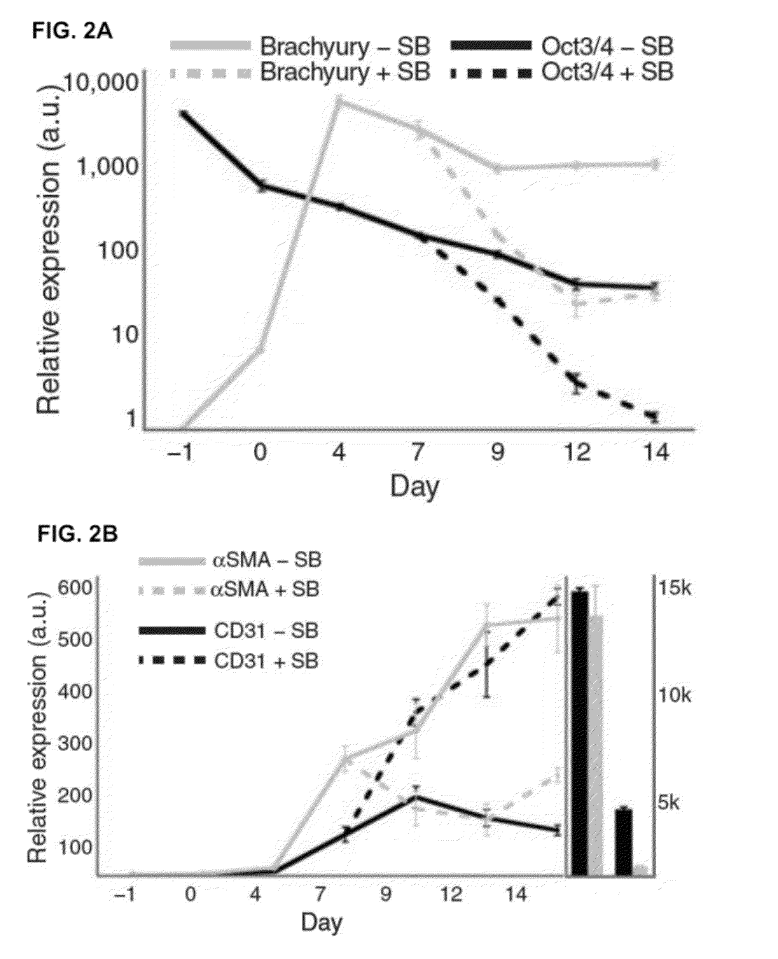

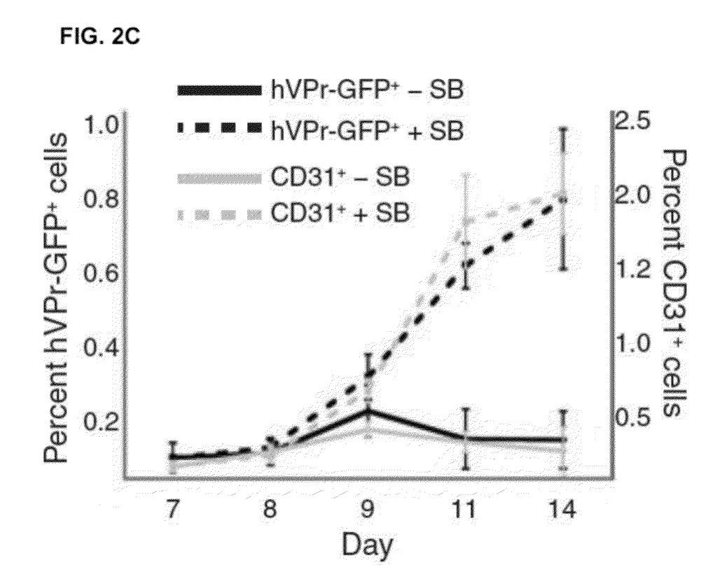

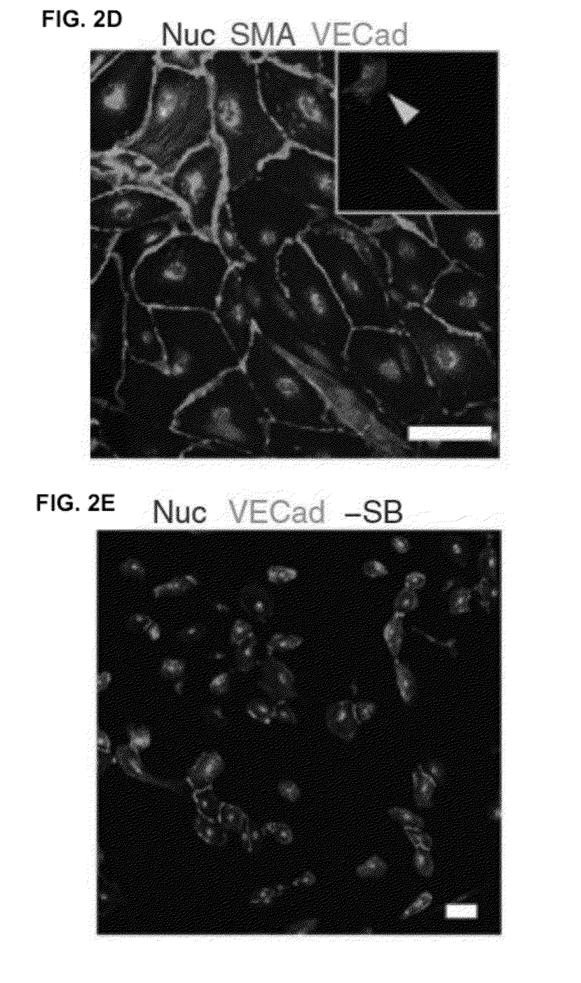

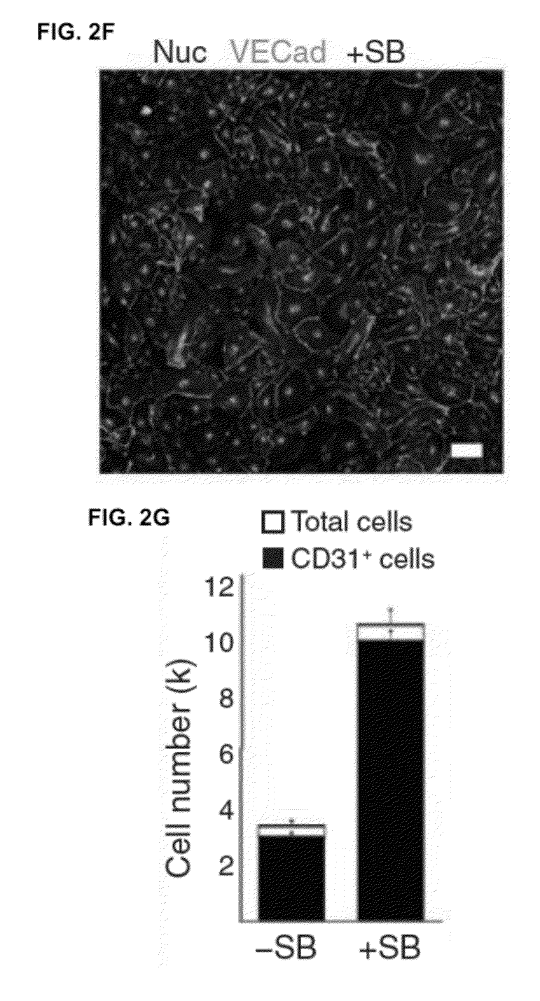

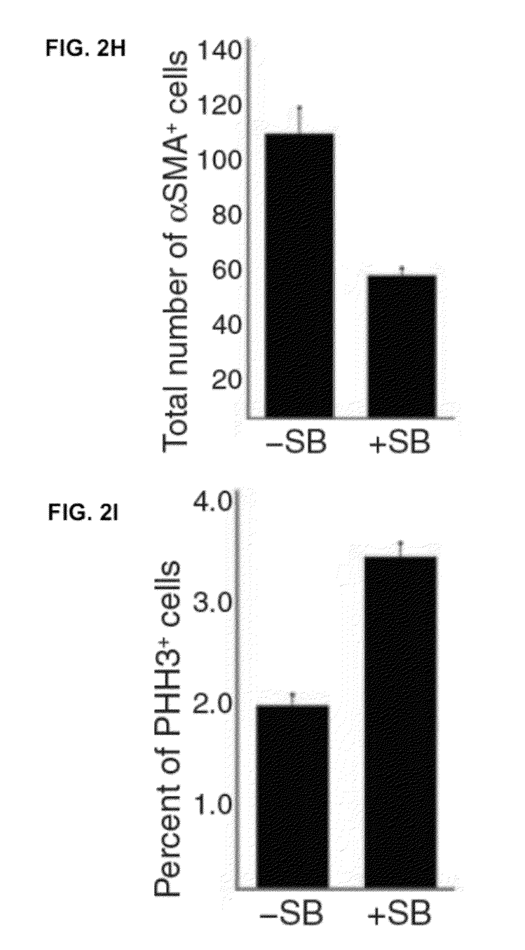

Kinetic analysis of differentiation in the presence or absence of TGFβ-inhibition revealed a shift in global transcriptional profile from pluripotent (Oct3/4⁺, FIG. 2A) to vascular (CD31⁺, FIG. 2B-C) phenotype via a mesodermal intermediate (brachyury⁺, FIG. 2A). Addition of SB431542 accelerated the reduction of both Oct3/4 and brachyury, and promoted a significant increase in hVPr-GFP⁺CD31⁺ ECs beginning at 9 days, while reducing expression of SMA (FIG. 2B-C). Isolated ECs cultured in the absence of TGFβ-inhibition retained high expression of CD31 but surprisingly, hVPr-GFP+CD31+ derivatives also expressed α-SMA, indicating that these endothelial cell-like cells had not assumed a terminally committed vascular fate.

Expression of α-SMA in hESC-derived ECs suggested a degree of plasticity that is not present in terminally differentiated ECs (HUVEC, FIG. 2B). Indeed, extended culture of hESC-derived ECs in the absence of TGFβ-inhibition yielded a significant number of cells coexpressing VE-cadherin and α-SMA (FIG. 2D). One explanation for the increased percentage of ECs in SB431542 stimulated cultures is maintenance of the vascular committed state following specification. To test the capacity for TGFβ-inhibition to promote expansion of pure populations of hESC-derived ECs, day 14 differentiation cultures were dissociated and ECs were isolated and expanded for an additional 5 days with or without SB431542 (phase 2, FIG. 2E-I). SB431542-treated cultures yielded more cells in the 5-day culture period, and a higher percentage of the total population retained a α-SMA⁻ CD31⁺VEcadherin⁺ phenotype (FIG. 2E-H). In addition to preserving the vascular phenotype, SB431542 also increased cell proliferation, as indicated by a higher percentage of Phospho-HistoneH3⁺ (PHH3) mitotic ECs (FIG. 2I).

In aggregate, TGFβ inhibition in phase 1 and 2 resulted in 36-fold expansion in the total number of vascular-committed hESC-derived ECs with a ratio of 7.4 ECs generated from every one hESC input over the course of 20 days, compared to 0.2 ECs per input hESC derived from control culture conditions (FIG. 2J). Furthermore, similar levels of expansion of hESC-derived ECs were achieved in 4 additional hESC lines and one induced pluripotent stem cell line using the same protocol except that either SB431542 or soluble TGFβRII receptor decoys was used interchangeably to inhibit activation of the activin/nodal branch of TGFβ superfamily signaling. These results demonstrate that the effect of TGFβ inhibition shown here is applicable to other pluripotent cells.

### Example 5

### Transcriptional Profile of hESC-Derived ECs

To define the vasculogenic transcriptional signature of hESC-derived ECs at different time points during phases 1 and 2, Affymetrix microarray analyses were conducted on several hESC-derived populations and mature cell types, including day 14 EBs differentiated with angiogenic cytokines; phase 1 purified ECs (day 14) differentiated with TGFβ-inhibition; phase 2 purified ECs, isolated at day 14 and cultured for an additional 10 days with TGFβ inhibition; along with HUVEC, SMCs and CD34⁺ hematopoietic cells isolated from umbilical cord and cord blood. Importantly, the yield of freshly isolated phase 1 ECs in the absence of TGFβ-inhibition was insufficient for microarray analyses, underscoring the value of the method disclosed herein for generating sufficient expanding (phase 1) and vascular-committed (phase 2) ECs for molecular profiling.

Phase 1 hESC-derived ECs showed increased levels of genes typical of arterial-like EC identity (VEGFR2, VEGFR1, Id1, CD31, CD34, VE-cadherin, vWF, thrombomodulin, EphrinB2, E-selectin), but not lymphatic ECs (Prox1, Podoplanin). Markers associated with vascular progenitor cells, including CD133 and Id1 (Gehling et al., Blood 95: 3106-3112 (2000); Kelly et al., Arterioscler. Thromb. Vasc. Biol. 29: 718-724 (2009); Peichev et al., Blood 95: 952-958 (2000); Rafii et al., Science 319: 163-164 (2008); Gao et al., Science 319: 195-198 (2008); Lyden et al., Nat. Med. 7: 1194-1201 (2001)), were also highly expressed in phase 1 ECs and down-regulated upon in vitro culture; and transcription factors expressed primarily in committed ECs, including HoxA9 (Rossig et al., J. Exp. Med. 201: 1825-1835 (2005)), were not expressed in phase 1 ECs. Accordingly, a global vasculogenic expression profile of hESC-derived ECs is defined by a VE-cadherin⁺VEGFR2^{high}Id1^{high} Thrombomodulin^{high}EphrinB2⁺CD133⁺HoxA9⁻ phenotype, while mature ECs can be identified as VE-cadherin⁺VEGFR2^{low}Id1^{low}EphrinB2⁺CD133⁻HoxA9⁺ phenotype.

### Example 6

### Id1 Mediated the Pro-Angiogenic Effect of TGFβ-Inhibition

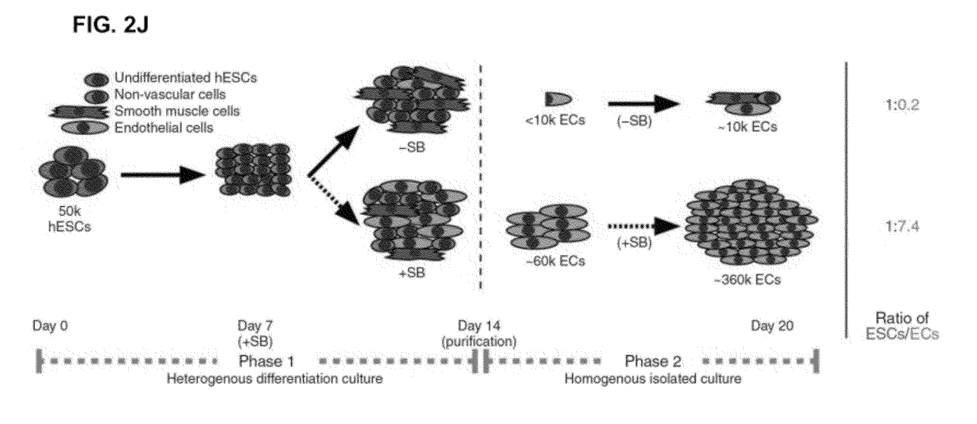

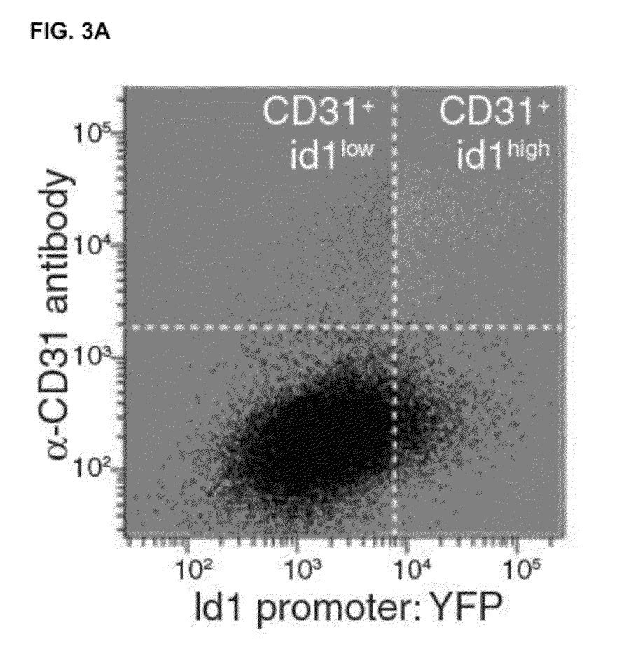

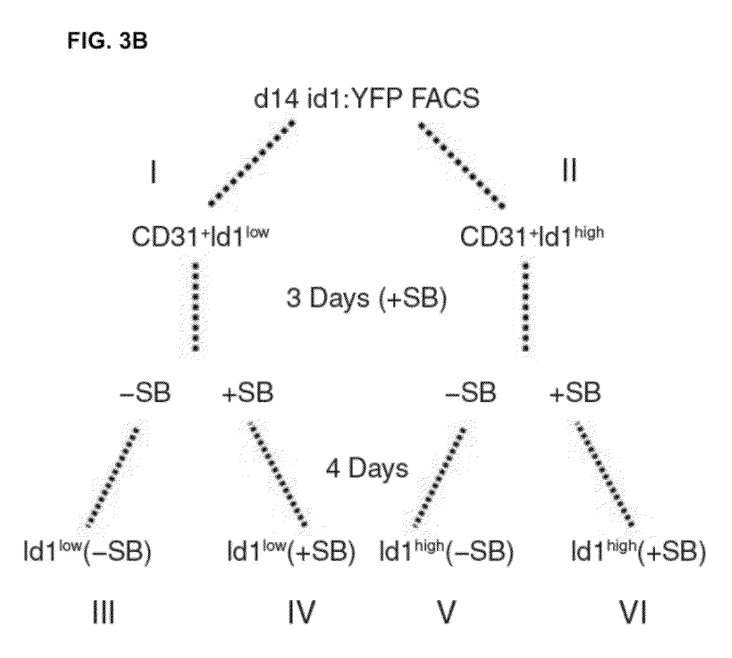

Id1 was one of numerous transcription factors upregulated in phase 1 ECs. Because Id1 had been shown to modulate differentiation and maintenance of vascular cell fate (Ruzinova et al., Trends Cell Biol 13: 410-418 (2003)), experiments were designed herein to test whether Id1 mediated the pro-angiogenic effect of TGFβ-inhibition. To track Id1 expression in live hESC differentiation cultures, a stable BAC transgenic hESC-line expressing yellow fluorescent protein driven by the Id1-promoter (Id1-YFP) (Example 3 herein) was used (FIG. 3A-C). Differentiated ECs were isolated at day 14 from Id1-YFP cultures (FIG. 1d), sub-fractionating the CD31+ population into Id1-YFP high- and low-expressing cells, and these populations were serially expanded for seven days with and without the TGFβ-inhibitor (FIG. 3B). Flow cytometric analysis of these cells revealed a direct relationship between up-regulation of Id1 expression and TGFβ-inhibition (FIG. 3C). Notably, although SB431542 increased the percentage of the CD31⁺ population, the mean fluorescence intensity of CD31 on these cells was decreased, relative to unstimulated cells. These data suggested that TGFβ-inhibition increased expansion of hESC-derived ECs by maintaining high levels of Id1 expression and preserving an immature proliferative phenotype.

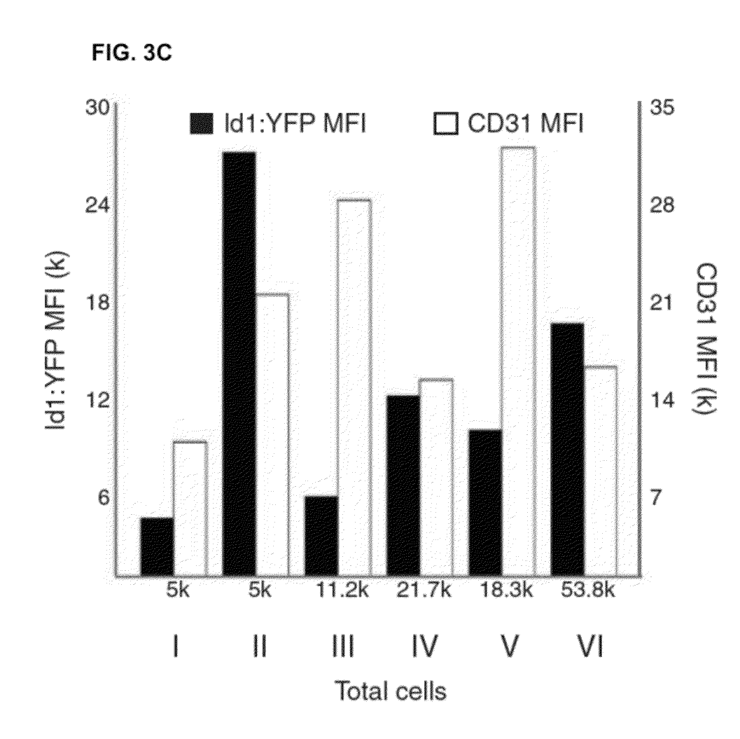

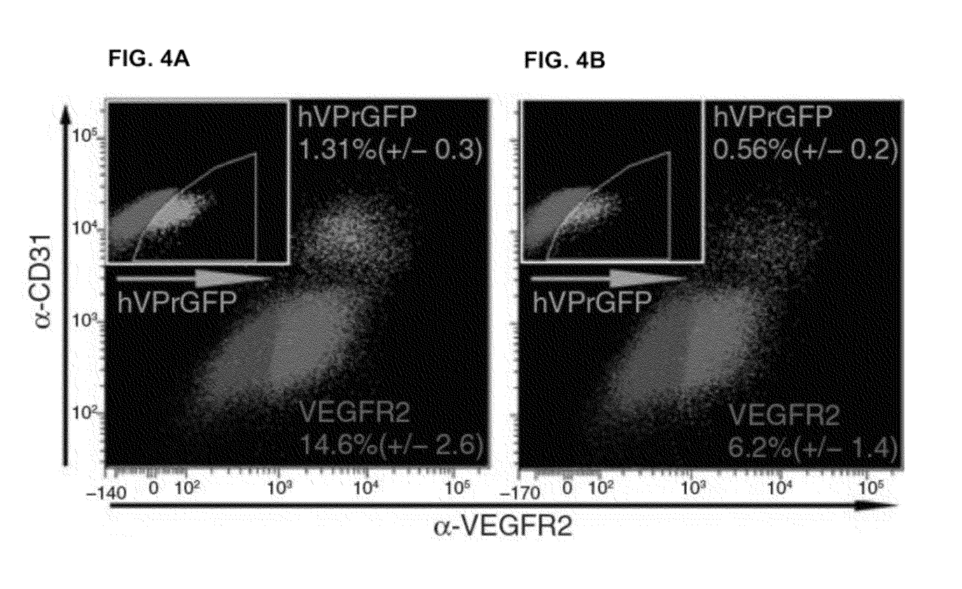

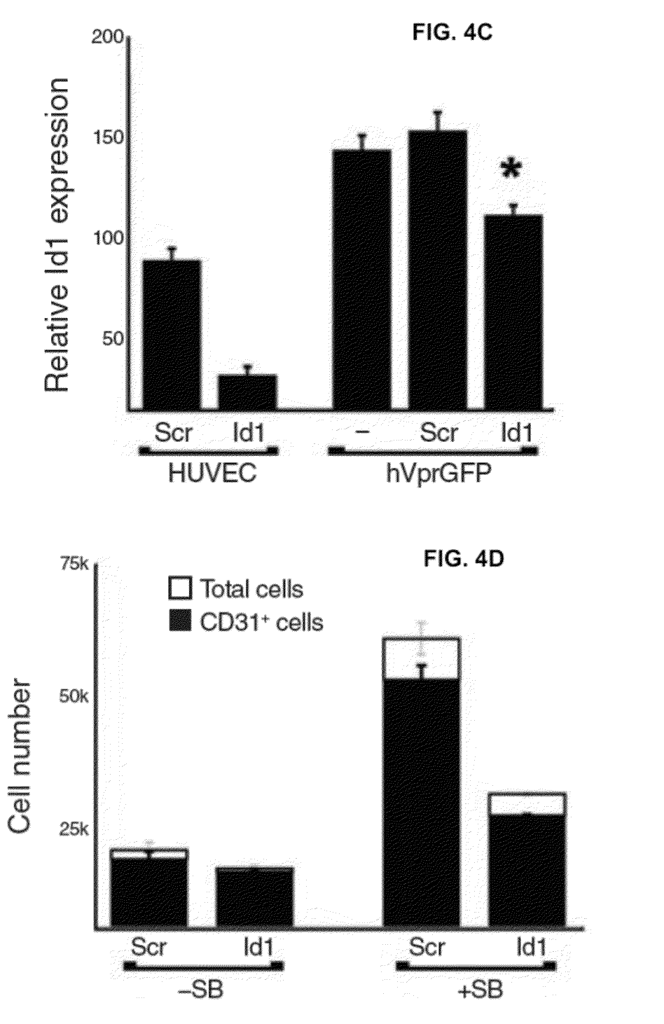

In order to determine the requirement for Id1 in mediating EC commitment, hVPr-GFP+ cells were tranduced with lentiviral short hairpin (sh) RNA targeted against the Id1 transcript (FIG. 4A). The Id1 and Control (Ctl) shRNA lentiviral constructs were obtained from Open Biosystems and viral particles were assembled according to the manufacturer's recommendations (pLKO Lentiviral Packaging System). The Id1 and Ctl shRNA constructs were used as described in Example 1 to stably transduce freshly isolated human VPr-GFP-hESCs, HUVECs, and freshly isolated (at day 14) hVPr-GFP⁺ cells. The Id1 ShRNA treated VPr-GFP-hESCs were differentiated according to the protocol shown in FIG. 1D and assessed at day 14 for the prevalence of VEGFR2⁺ (blue) and hVPr-GFP⁺ (green) cells. The relative Id1 transcript levels of the Control and Id1 specific shRNAs treated HUVECs and freshly isolated (at day 14) hVPr-GFP⁺ cells were measured following 3 days. Control and Id1 specific shRNAs treated freshly isolated hVPr-GFP⁺ cells were cultured in the absence or presence of SB431542 for 5 days. The total cell number and percentage of CD31⁺ cells was measured by flow cytometry.

In the presence of SB431542, knockdown of Id1 reduced the incidence of VEGFR2⁺ cells and hVPr-GFP⁺ ECs at day 14. When the Id1 shRNA construct was introduced following isolation of the hVPr-GFP⁺ fraction (FIG. 4C), it elicited a marked decrease in CD31⁺ ECs following 5 days of SB431542 treatment (FIG. 4D). These results identified TGFβ-inhibition mediated Id1 upregulation as a primary effector in promoting EC expansion and maintaining long-term vascular identity.

### Example 7

### In Vivo Test of hESC-Derived ECs in Mice

To demonstrate that the ECs generated herein could form functional vessels, purified hVPr-GFP⁺ cells from day 14 differentiation cultures were grown for additional 8 days in the presence of SB431542. These ECs showed high proliferative potential (>10 population doublings), and generated homogenous hVPr-GFP⁺VE-cadherin⁺ monolayers with retention of hVPr-GFP fluorescence at the single cell level. These cells were subcutaneously injected in Matrigel™ plugs into nonobese (NOD)/severe combined immunodeficient (SCID) mice and 10 days later, extracted from animals that had been injected intravenously with lectin. In Matrigel™ plugs, hVPr-GFP⁺ cells co-localized with lectin⁺ cells, forming chimeric vessels along with host cells. These data indicated that the ECs generated by the methods of this invention can function in vivo.

### Example 8

### Discussion

A prerequisite to therapeutic vascularization using hESC-derived cells is generation of abundant durable ECs that upon cellular expansion maintain their angiogenic profile without differentiating into non-EC types. The data disclosed herein prove that differentiation of hESCs into a large number of stable and proliferative ECs can be achieved by early stage TGFβ-mediated mesoderm induction followed by TGFβ-inhibition beginning at day 7 (phase 1) and following isolation at day 14 (phase 2). Employing this approach, a 36-fold net expansion of committed ECs was achieved. This increased yield of hESC-derived ECs afforded analyses of their transcriptional profile, revealing a unique molecular signature that sheds light on the regulatory influences that govern embryonic vasculogenesis. Id1 was found to act downstream of TGFβ-inhibition to augment EC yield by increasing proliferation and preserving vascular commitment. These studies establish TGF modulation of Id1 expression as a determinant of hESC-derived EC identity and set the stage for large-scale generation of authentic long-lasting human ECs for therapeutic vascularization.

The use of vascular-specific hVPr-GFP and Id1-YFP hESC reporter lines in small molecule screens enabled the elucidation of the TGFβ-inhibitor SB431542 as a key stimulus for human EC differentiation and proliferation in serum-free conditions. In murine ESCs, TGFβ and serum factors promote smooth muscle cell differentiation, while inhibition of this pathway promotes formation of CD31⁺ cells (Watabe et al., J. Cell Biol. 163: 1303-1311 (2003)). The data disclosed herein showed that stage-specific TGFβ-inhibition, beginning at a point following TGFβ-mediated mesoderm induction (for example from day 7), increased mitotic index and maintenance of hESC-derived ECs via upregulation of Id1 expression. Differentiation of hVPr-GFP hESCs with TGFβ-inhibition generated ECs at yields 10-fold greater than cells differentiated with angiogenic factors alone, and following purification, TGFβ-inhibition supported EC expansion for more than 10 population doublings, while retaining the angiogenic surface phenotype. The capacity for TGFβ-inhibition to augment EC yield in both differentiating (phase 1), and purified (phase 2) cultures, resulted in a 36-fold increase in the absolute number of hESC-derived ECs, with 95% of the population maintaining EC identity. As such, this disclosure has established a means of generating a homogeneous population of stable ECs in ratios that significantly exceed hESC input, and thus addressed a major obstacle to therapeutic vasculoplasty.

Expression of Id1 has been shown to inhibit cell differentiation and growth arrest in multiple cell types (Jankovic et al., Proc. Natl. Acad. Sci. 104: 1260-1265 (2007)) and the TGFβ signaling pathway, by way of the effectors Smad3 and ATF3, has been shown to repress Id1 promoter activity (Kang et al., Mol. Cell. 11: 915-926 (2003)). These data disclosed herein point toward a biphasic role for TGFβ signaling during vasculogenesis, whereby early activation of this pathway is required for specification of mesodermal progenitors, and inhibition following vascular commitment functions to increase mitotic index and prevent the loss of endothelial identity. The methodologies disclosed herein for vascular monitoring and differentiation permit identification of as yet unrecognized vasculogenic and angiogenic modulators that can be employed in pre-clinical studies aimed toward the cell based therapeutic revascularization of ischemic tissues.

What is claimed is:

1. A method for developing human endothelial cells (ECs) from human pluripotent cells in culture, comprising:
forming embryoid bodies (EBs) from said pluripotent cells;
culturing the EBs under conditions sufficient for mesoderm induction;
culturing the cells under conditions that promote vascular differentiation and generation of ECs in the cell population; and
further culturing the cells in the presence of a TGFβ signaling inhibitor to expand ECs in the cell population.

2. The method of claim 1, wherein said pluripotent cells are embryonic stem cells (ESCs).

3. The method of claim 1, wherein said pluripotent cells are induced pluripotent cells.

4. The method of claim 2, wherein EBs are formed by culturing the human ESCs in media devoid of FGF-2 and supplemented with a BMP.

5. The method of claim 1, wherein the step of culturing the EBs under conditions sufficient for mesoderm induction comprises culturing the EBs in media supplemented with an activin and a BMP, and further culturing in media additionally supplemented with FGF-2.

6. The method of claim 5, wherein the activin is activin A, the BMP is BMP4.

7. The method of claim 6, wherein the EBs are cultured for 2 days in the presence of activin A and BMP4, and further cultured for 2-3 days in the presence of activin A, BMP4 and FGF-2.

8. The method of claim 1, wherein the step of culturing the cells under conditions that promote vascular differentiation and generation of ECs comprises culturing the cells on an adherent substrate in media supplemented with VEGF-A, a BMP and FGF-2.

9. The method of claim 8, wherein the cells are cultured for 3-4 days.

10. The method of claim 1, wherein said TGFβ signaling inhibitor is an inhibitor specific for the type I TGFβ receptors.

11. The method of claim 10, wherein said inhibitor inhibits signaling mediated by ALK4, ALK5, and ALK7.

12. The method of claim 10, wherein said inhibitor is an inhibitor of at least ALK5.

13. The method of claim 10, wherein said inhibitor is a polypeptide comprising a soluble form of a type I TGFβ receptor, an antibody directed to a type I TGFβ receptor, or a small molecule compound.

14. The method of claim 10, wherein said inhibitor is a small molecule compound selected from SB-431542, A 83-01, D 4476, LY 364947, SB 525334, SD 208, and SJN 2511.

15. The method of claim 14, wherein said inhibitor is SB-431542.

16. The method of claim 1, wherein ECs are expanded in the presence of said TGFβ signaling inhibitor, VEGF-A and FGF-2.

17. The method of claim 1, further comprising isolating expanded ECs from the cell population.

18. The method of claim 17, further comprising culturing the isolated ECs in media supplemented with said TGFβ signaling inhibitor, VEGF-A and FGF-2.

19. A substantially pure population of hESC-derived ECs, wherein said ECs are characterized by expression of surface markers, VE-cadherin, CD31 and VEGFR2.

20. A composition comprising the substantially pure population of ECs of claim 19 and at least one pharmaceutically acceptable carrier or diluents.

21. A method for repairing injured tissue in a human subject, comprising administering to the subject the composition of claim 20 to promote vascularization in said tissue.

22. A method for treating a tumor in a human subject, comprising administering to the subject the composition of claim 20, wherein said ECs are engineered to deliver an anti-tumor agent, and upon administration, said ECs form vessels into said tumor.

23. A human ESC reporter cell line, said cell line stably transfected with a nucleic acid molecule coding for a fluorescent protein, operably linked to the promoter region of the VE-cadherin gene.

24. The reporter cell line of claim 23, wherein said fluorescent protein is selected from the group consisting of green fluorescent protein (GFP), blue fluorescent protein (BFP), mOrange fluorescent protein, mCherry fluorescent protein, and yellow fluorescent protein (YFP).
